(19) United States
(12) Patent Application Publication
(10) Pub. No.: US 2010/0239646 A1
(43) Pub. Date: Sep. 23, 2010

(54) Sublingual methotrexate and methotrexate patches

(76) Inventor: Madhavan G. Nair, Mobile, AL (US)

Correspondence Address: Madhavan G. Nair, 7005 Charleston Oaks Drive North, P.O. Box 16144, Mobile, AL, 36616, US

(21) Appl. No.: 12/381,994

(22) Filed: Mar. 18, 2009

(51) Int. Cl. A61K 9/70; A61P 19/02; A61K 31/4985; A61P 11/06; A61P 17/06; A61P 1/00; A61P 1/16; A61P 35/00; A61P 35/02; A61P 9/10; A61P 9/00; A61P 25/28

(52) U.S. Cl. 424/449; 514/249

(57) ABSTRACT

Several major new inflammatory diseases that respond to methotrexate are identified in this Invention. These newly identified indications that respond to methotrexate include a number of cardiovascular diseases, coronary heart disease and stroke. To optimize and improve therapeutic effectiveness, therapeutic responses and to minimize toxicity new sublingual dosage units and patches of methotrexate are taught in this Invention for direct delivery of the drug to the blood stream bypassing absorption through the human gut. The practice of this invention as provided herein should result in significant improvement of the therapeutic index of methotrexate in the treatment of a number of painful, crippling, disfiguring, and fatal diseases. This Invention therefore addresses an unmet need to treat the above diseases for which there are no curative treatments are available at the present time. The use of sublingual methotrexate or methotrexate patches to prevent, treat or reverse cardiovascular diseases, coronary heart disease and stroke is new and the practice of this invention should result in very significant therapeutic improvements and commercial benefits.

### FIELD OF INVENTION

### General Field

### Indications, Therapies, Formulation and Administration of Antifolates

New therapeutic indications and formulation of methotrexate to overcome current concerns of its inter-patient variability of toxicity, therapeutic responses and therapeutic efficacy in patients suffering from inflammatory diseases, autoimmune diseases and malignant and benign neoplastic diseases are described. The invention described herein teaches the use of methotrexate for the treatment, prevention and reversal of cardiovascular diseases, coronary heart disease and stroke.

### DESCRIPTION OF INVENTION

Methotrexate (MTX) is a well-known antifolate drug that interferes with tetrahydrofolate utilization [M. G. Nair. “ANTIFOLATES”. In “Cancer Growth and Progression—Cancer Control in Man”. Chapter 10. H. E. Kaiser (Ed). Kluwer Academic Publishers (1990); M. G. Nair. “Chemistry of Antifolates”. In “The Chemistry of Antitumor Agents”. D. E. V. Wilman (Ed) Blackie and Sons (Lond); Chapman and Hall (USA) Chapter-7 (1990)] at various stages of the complex one carbon metabolism mediated by folate coenzymes. Inherently and by mechanisms of action methotrexate is very toxic at significantly higher concentrations relative to their therapeutically effective and safe plasma levels.

Chemotherapy of neo-plastic diseases, diseases secondary to uncontrolled or abnormal cell proliferation, inflammatory diseases with or without an auto-immune component with methotrexate utilizes parenteral administration to circumvent fluctuations in plasma drug concentrations secondary to variations in bio-availability when other dosage forms are prescribed (M. G. Nair. Antifolate drugs in Chemotherapy (review). Drug Discovery Today 4: 492-494 (1999). For instance a ten percent variation in bioavailability by the oral route may cause similar fluctuation in plasma drug levels and a ten percent increase relative to the optimal therapeutically effective concentration of the drug in plasma may lead to dangerous and undesired biological consequences. Since oral administration of antifolates are infrequently used for most common antifolate indications, methotrexate, also known as rheumatrex is preferred to be administered by the oral dosage form for simplicity and convenience in very low doses for the treatment of rheumatoid arthritis and other auto-immune and inflammatory diseases [(G. S. Alarcon, M. O. Castaneda, M. G. Nair, A. Berrocal, E. Paz, W. J. Koopman, and C. L. Krumdieck. Controlled trial of methotrexate versus 10-deazaaminopterin in the treatment of rheumatoid arthritis. Annals. Rheumatic Diseases, 51, 600 (1992); G. Alarcon, O. Castaneda, W. J. Koopman, C. L. Krumdieck, and M. G. Nair. 10-Deazaaminopterin: A New Arthritis Remittive Drug. U.S. Pat. No. 5,030,634 (1991)] that require only relatively low doses. Methotrexate usually does not present significant fluctuations in bioavailability during treatment of rheumatoid arthritis due to the very low average dosage of about 15 mg/week combined with its inherently high bioavailability from approximately 28 to 88 percent. [Arthritis and Rheumatism. 60, 1-4 (2009)]. However even for methotrexate, low bioavailability of less than 30 percent in certain patient populations, even a 5% to 25% variation in absorption among individual patients can lead to serious consequences of toxicity or lower plasma levels of the drug that results in non-responsiveness to therapy.

It is well documented that some patients with rheumatoid arthritis do not respond to methotrexate [methotrexate non-responders] at doses that were found to be effective in a majority of patients (O. Castaneda and M. Gopal Nair. Controlled trial of methotrexate versus CH-1504 in the treatment of Rheumatoid Arthritis. The J. Rheumatology. 33, 862-64, 2006). It is also known that some patients treated with Rheumatrex (methotrexate) at normally accepted safe and therapeutically effective doses show undesirable toxicities compelling them to withdraw the drug. Although several theories are promulgated to explain the above observations, likely causes may also include sub-optimal and super-optimal plasma drug levels secondary to individual variations in drug absorption from the gut when oral dosage from of the drug is used.

Sublingual dosage forms of methotrexate and methotrexate skin patches (trans dermal skin patches) claimed in this Invention are unprecedented and such dosage forms and methods of delivery of methotrexate are expected to induce excellent and safe biological responses (secondary to stable and predictable drug plasma concentrations) and therapeutic utility in treating several dozens of diseases that respond to antifolate therapy. In addition, this invention teaching the use of methotrexate for the prevention, control, treatment and reversal of coronary heart disease, cardiovascular disease, stroke and related inflammatory diseases is new and the practice of which would have enormous medical utility and potential commercial benefits by virtue of more effective and less toxic therapeutic outcome.

### BACKGROUND OF INVENTION

### Prior Art

Sublingual formulations of a number of therapeutic agents and difficulty absorbing vitamins (verapamil, morphine, vitamin B12 and steroids) are currently in use [L. Lea, (www.positivehealth.com/permit/Articles/Colon%20Health/lea13.htm)]. Sublingual formulations of compounds that are relevant to this invention are only very few and they are vitamin derivatives (folic acid and 5-formyltetrahydrofolic acid). Others are certain vitamins that include B12 (cyanocobalamin derivatives) [Survival Enterprises, Idaho, U.S.A. No Shot ^{R}.B12, B6 and Folic acid]. Sublingual formulations of antifolates to circumvent problems of bioavailability from oral dosage forms are not utilized. Therapies with sublingual dosage forms of methotrexate to ameliorate any diseases that respond to antifolates have no precedence. In addition an Internet Search on the topic “sublingual methotrexate” using commonly used search engines failed provide any relevant references on this topic. The concept and reduction to practice of sublingual administration of methotrexate to overcome methotrexate resistance, methotrexate toxicity, or non-responsiveness and to create uniform and predictable methotrexate plasma levels is unexpected and unprecedented. Methotrexate is not currently used as a medication to treat, prevent or reverse coronary heart disease, cardiovascular diseases or stroke. The above serious and often fatal diseases are new indications identified in this Invention that can be subjected to successful therapeutic interventions by methotrexate.

### ADVANTAGES OF THIS INVENTION

This invention teaches a method of administering methotrexate directly via the blood vessels in the mouth (under the tongue and cheeks) that facilitates efficient, direct and quick absorption of the drug to the blood stream bypassing the gastrointestinal (GI) tract. Sublingual administration of drugs and vitamins offers an attractive and efficient alternative for drug delivery in patients who suffer from hyperactive stomach, ulcers, indigestion, celiac disease, stomach cancer, alcoholism and any abnormalities of the GI tract that interferes with absorption. These conditions may also include Crohn's disease, Inflammatory Bowel Disease (IBD) and other inflammatory diseases affecting the GI tract. Sublingual antifolate delivery is predictive of drug blood levels achievable by this route and is independent of any GI tract abnormalities. Sublingual drug delivery stabilizes and increases the bioavailability. No carrier mediated or active transport of the drug is needed when sublingual dosage forms are used to achieve uniform, dose dependent and predictable drug levels in the plasma The drug is first absorbed directly to the sublingual artery that is connected to the lingual artery and finally enters the blood stream via the common carotid artery. Absorption occurs by passive diffusion and not by active (energy requiring) or carrier mediated transport. It is also likely that absorption of sublingual formulation of the drug may take place by endocytosis or pinocytosis. This method of sublingual administration of methotrexate thus overcomes any bioavailability concerns associated with the use of its oral dosage units. Often it is only necessary to administer the drug in significantly lower doses by sublingual method relative to the doses required by the oral dosage units as a result of increased bioavailability of the drug via the sublingual route. In fact it has been observed that only 20-25% of estradiol is need to be administered to women by the sublingual route to achieve the same desired bioavailability relative to the dosage used in the oral solid dosage unit of the hormone.

A significant and additional advantage with a classical antifolate such as methotrexate is an acid by virtue of having a glutamate moiety, capable of providing protons to the aqueous chime of the buccal cavity to induce vasodilatation resulting in enhanced absorption of the drug. The sublingual absorption of the classical antifolate, methotrexate would thus dramatically stabilize drug levels to predictable and uniform concentrations. Although a number of different types of therapeutic agents, vitamin and minerals are also administered by sublingual delivery, interestingly no antifolate compounds are currently administered by this method to treat human disorders that respond to classical antifolate therapies. This may be due to the fact that only methotrexate is currently under use in very low doses to treat chronic diseases such as rheumatoid arthritis and uvitis and very little is known about details of the mechanisms involved in methotrexate unresponsiveness and unexpected toxicity observed in some of the patients. Fluctuations in bioavailability secondary to differences in absorption of the drug from the gut and the resultant significant variations (high or low) in drug plasma levels among patients might account for these observations. Nevertheless, no attempts have yet been made to maintain the bioavailability, predicable and constant by changing the oral formulation of this very important drug, which is also known as Rheumatrex. Sublingual administration of methotrexate and other antifolates offers the alternative to address and alleviate the above problems of unresponsiveness and toxicity that are some times very serious secondary to changes in unpredictable drug plasma levels.

This inventions therefore teaches a method to circumvent unpredictable resistance, unresponsiveness and unexpected and some times very severe toxicities associated with the oral administration of methotrexate to treat common inflammatory and autoimmune diseases including but not limited to rheumatoid arthritis and uvitis.

Besides neoplastic diseases that are characterized by both benign and malignant abnormal cell proliferation, antifolate therapies are useful for the treatment of Inflammatory Diseases (A. Desai, and M. G. Nair. Evaluation of the anti-inflammatory activity and an alternate synthesis of a thiophene substituted 10-deazaaminopterin. Advances in Experimental Medicine and Biology: Chemistry and Biology of Pteridines and Folates. 338:425-428 (1993), Diseases caused by allergens and autoimmune immune diseases (M. G. Nair. Metabolically inert anti-inflammatory and anti-tumor antifolates. U.S. Pat. No. 5,912,251). Although the diseases that respond to antifolate therapies are numerous encompassing a large number of malignant and non-malignant cancers characterized by abnormal cell proliferation, inflammatory diseases, autoimmune diseases, diseases associated with allergic responses, new indications for methotrexate are identified in this Invention. The indications that can be treated with the sublingual administration of methotrexate described herein, include but not limited to Coronary Heart Disease (CHD), cardiovascular diseases (CVD), stroke and peripheral vascular diseases. Other methods to administer methotrexate are time released and dosage regulated patches, creams, jelly or gels. These time released dosage regulated patches may be worn over any part of the body that comes in contact with the skin to facilitate absorptive delivery. The creams, jelly or gels containing methotrexate may be placed inside a bandage or impregnated to a substance that forms a lining inside the patch. The technical design of the patch may be similar or modifications thereof the currently available patches for therapeutic delivery of steroids, steroid hormones and birth control medications. A list of the abnormalities that may be resolved with the sublingual dosage units of methotrexate described in this invention is provided below.

Inflammatory Diseases Caused by Allergens or Auto-Immune Response that Respond to Methotrexate Reported in this Invention:

Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma, Occupational asthma; Exercise induced asthma; Pediatric asthma; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhino sinusitis; Nasal polyposis; Laryngeal disorders; Peanut allergy; Eosinophillic esophagitis; Photo allergy; Photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodgkin lymphoma; Primary cutaneous lymphoma; Monocytic leukemia; Systemic monocytosis; (Connective Tissue disorders): SSc; SLE; PM-DM; Sjogren's; Juvenile chronic arthritis; JIA; Other JIA; Polyarticular JIA; (Hematological): Immune thrombocytopenic purpuria, Evan's; Auto-immune hemolytic anemia; pure red cell; pure white cell and others (Vasculitis): Wegener's; Behcet's; Takayasu; Macroscopic poly. Nodosa; Classical poly. Nodosa; Churg-Strauss; Others (Other Neurological): Mysthenia Gravis and many others described in EBMT Register. [D. Farge, EBMT Autoimmune diseases working party, March 2008. The Rheumatologist. 2: 21, 2008.]

Considering that the antifolate drug methotrexate has a theoretical bioavailability ranging from 20-80 percent when used as the oral dosage from. The solubility of methotrexate, like other classical antifolates bearing a terminal amino acid moiety is pH dependent and its bioavailability is thus sensitive to the pH variations in the gut of individual patients. Thus if all the patients taking oral dosage form of methotrexate has the ideal and optimal pH in the gut approximately 80 percent of the oral dosage of the drug will be absorbed. If the optimal therapeutic efficacy and response to the disease is based on this theoretical bioavailability, then all of the patients will respond to the therapy. Now if one assumes that one of these patients has a gut pH slightly lower than the optimal to reduce the availability by five percent and assuming that the oral dosage from has 10 mg drug, this patient will have only 7.5 mg of drug absorbed versus the optimal 8.0 mg. Like wise if the pH is slightly higher, then due to enhanced solubility or other factors that results in an increase of Bioavailability by five percent, 8.5 mg of the drug will be absorbed. These minor variations in bioavailability [7.5-8.5 with 8.0 being the optimal] should not detrimentally affect the therapeutic outcome of treatment of the disease with methotrexate in this patient group. Thus if the bio-availability of methotrexate is high enough in the patient population using the oral dosage form a 5-10% variations in drug absorption form the gut may not be significant enough to adversely effect the therapeutic outcome. In this example substitution of the oral dosage from with a sublingual dosage form of the drug will not result in significant changes in the therapeutic responses.

### PREFERRED EMBODIMENTS

### Example-1

This group of arthritic patients treated with methotrexate exhibited 20% absorption of methotrexate and the amount of drug in the oral dosage form is tailored to provide optimal therapeutic efficacy and responses calculated from this absorption datum. Since methotrexate is inherently a toxic compound that can cause severe and often unexpected toxic manifestations at significantly higher than optimal therapeutically effective plasma levels treatment of patients exhibiting low bio-availability pose certain significant problems and danger. If 10 mg of the drug is given by the oral route only 2.0 mg is absorbed. If the optimal therapeutic dosage is tailored based on this low absorption, then the therapeutically effective dose without manifestation of significant toxic effects would be an effective accumulation of 2.0 mg of the drug in the plasma. It is reasonable to assume for antifolates that an increase of 50% of drug level in plasma relative to the accepted safe level would lead to disastrous toxic consequences including severe bone marrow depression. Therefore an increase of bioavailability from 20 to 30 percent in this case would effectively increase drug concentration by 50% in the plasma. The 10 percent increase in Bioavailability may well be envisioned by slight pH changes in the gut of patients who may be taking antacids, or medicine to modulate acid secretions, taking other medications or unknown factors. The present invention addresses this very crucial safety issues and provides a means to stabilize drug concentration in plasma to predictable levels by replacing oral dosage form of methotrexate with the sublingual dosage form, the absorption of which is independent of the biochemical pharmacology of human gut.

### Example-2

Based on ADMEP (Absorption, Distribution, Metabolism, Excretion, Pharmacokinetics) studies conducted with an antifolate compound in animals and humans during pre-clinical investigations and Phase-I-II clinical trials it was determined that an absorption of 1.0 mg of the drug from the gut to plasma three times a day from divided oral dosage units is optimal (3.0 mg/24 hours) in treating the antifolate responsive disease without causing significant toxicity. It was also determined in this hypothetical study that a concentration of 1.5 mg of the antifolate from the gut to the plasma three times a day (4.5 mg/24 hours) results in unacceptable GI and bone marrow toxicity. The bioavailability of this drug was only 10 percent as determined from a Phase-1-a clinical trial in healthy volunteers. Based on the calculated bioavailability figure obtained from healthy humans a solid oral dosage form of 10 mg per dosage unit was formulated to assure optimal plasma drug delivery (1.0 mg×3=3.0 mg/24 hours) to patients. The Bioavailability of this new medication was pH dependent due to solubility reasons and therefore it was possible that fluctuations in Bioavailability might occur in certain patients as described in example-1. Thus in certain patients taking antacids or drugs such as certain prostaglandins to reduce acid secretion or other unknown factors the pH of the gut might increase to enhance the solubility and thereby increasing the bio-availability to the 15 to 20% range. Therefore in this particular instance an increase in bioavailability from 10 to 15% might result in dangerous plasma levels of the drug (1.5 mg×3=4.5 mg/24 hours) with unacceptable toxicities and serious side effects.

Likewise a drop in gut pH in certain patients due to various known or unknown reasons can reduce the bioavailability of this drug from 10 to as low as 5% or even lower. This situation may lead to sub-optimal concentration of the drug causing unresponsiveness and ineffective therapy using the 10 mg oral dosage unit form.

It is therefore of paramount importance that the plasma levels of this antifolate be kept essentially constant during the entire duration of therapy. As shown in the above examples this consistency is impossible to accomplish using the solid oral unit dosage form of an antifolate drug with low bioavailability in all patients.

### Example-3

### Methotrexate-Misadventure

A clinical pharmacist reviewing a patient's own medications discovered the MTX misadventure. A bottle labeled as ‘2.5 mg methotrexate tablets’ contained actually the 10 mg tablets. The patient had taken six tablets in two weeks (6×10 mg=60 mg) weekly instead of her usual 15 mg (6×2.5 mg) weekly dose prescribed to treat rheumatoid arthritis. Further investigation revealed that the patient's general practitioner had prescribed MTX 10 mg tablets with the instructions ‘Take one and a half tablets weekly’. Her local pharmacy dispensed the 10 mg tablets and labeled the bottle correctly. Upon arrival at home the patient, out of habit, emptied the contents of the new prescription into an old bottle of MTX tablets which bore the label ‘Methotrexate 2.5 mg tablets. Take 6 tablets weekly’. The patient was unaware of the change in tablet strength and directions, and was subsequently taking the incorrect dose resulting in odynophagia, dysphagia and oesophagitis.

Odynophagia and dysphagia relief was obtained using antacids. Oesophagitis was secondary to MTX. Calcium folinate 15 mg orally four times a day was commenced. Within 24 h the odynophagia had improved and the patient was well enough to be discharged. [S. Yeoh and J. Siderov in the journal Rheumatology. 40: 230-232 (2001)]

Methotrexate misadventure is not a new problem [1-3]. The major risk outlined in these reports relates to frequency of dosing. The dose of MTX used for the treatment of different diseases varies significantly. In the treatment of rheumatoid arthritis and psoriasis, the dose is often once each week, either as a single or divided dose. “There have been instances where this weekly dose has, in error, been taken either daily or on several days each week, resulting in severe unwanted effects, including neutropenia, hepatotoxicity and bone marrow suppression. [1. Brown M A, Corrigan A B. Pancytopenia after accidental overdose of methotrexate. Med. J. Aust. 1991; 155:493-4; 2. Lomaestro, B M; Lesar, T S; Hager T P. Errors in prescribing methotrexate [letter]. J Am Med Assoc 1992; 268:2031-2; 3. Isdale, A H; Hordon, L D. Daly M. Methotrexate mishap [letter]. Br J. Rheumatol 1994; 33:503-4].

### Example-4

O. Castaneda and M. Gopal Nair conducted the clinical trial of a metabolism-blocked antifolate Mobileterx [M-Trex] in patients with rheumatoid arthritis to evaluate its efficacy relative to the well-known antifolate drug methotrexate. In this pilot study [O. Castaneda and M. G. Nair. [The J. Rheumatology. 33, 862 (2006)] it was observed that as the treatment progressed the plasma concentration of the inflammatory mediator, C-Reactive Protein (CRP) which is also a marker of inflammation consistently decreased. [See Table of M-TREX Responses below]

In patients, sublingual methotrexate and methotrexate patches may be used in conjunction or concurrently with TNF-α inhibitors [enbrel, humira, remicade], non-steroidal antiinflammatory agents (NSAIDs], corticosteroids, hydroxychloroquine, sulfasalazine, leflunomide, neural, auranofil and anakinra.

M-TREX RESPONSES

| | 4 wks | 8 wks | 12 wks | 16 wks |
|---|---|---|---|---|
| Pain | 4.1 | 2.8 | 2.2 | 1.8 |
| HAQ | 1.8 | 1.6 | 1.4 | 1.4 |
| T. Joints | 10.2 | 7.3 | 5.8 | 5.8 |
| S. Joints | 5.6 | 3.5 | 3.0 | 2.9 |
| Pa. GA | 6.0 | 6.0 | 5.0 | 3.0 |
| Ph. GA | 6.2 | 5.0 | 4.5 | 3.0 |
| ESR | 52.7 | 32.7 | 38.3 | 38.6 |
| CRP | 22.1 | 20.7 | 17.7 | 13.6 |

CRP levels are currently correlated with the incidence and progression of cardiovascular diseases (CVD), coronary heart disease (CHD) and stroke. The higher the CRP levels the higher the risk of the incidence of CVD, CHD, Stroke and related diseases. The unexpected and new finding of the ability of M-Trex to reduce CRP levels during treatment and previous long-term studies with methotrexate led to the present invention of methotrexate as a therapeutic agent for the prevention, control or reversal of CVD, CHD, Arterioscleroses, Stroke and related diseases in humans and other warm blooded animals. At the present time no antifolates including methotrexate are used as a prophylactic agent for the prevention of the onset or as a therapeutic agent for the control, treatment or reversal of these very serious and often fatal inflammatory diseases. Therefore it is realized that methotrexate is capable of exhibiting therapeutic properties that address an unmet but extremely important need to treat and/or prevent a series of serious diseases such as CHD, CVD and stroke that have fatal or crippling consequences in affected human beings. The sublingual formulation or patches of methotrexate described as Inventions herein, by virtue of predictable and direct delivery to blood stream bypassing absorption via the human gut, thus should result in improved therapeutic efficacy in the treatment of these diseases.

### Example-5

### Resistance and Toxicity of Methotrexate

During conventional therapy of rheumatoid arthritis with methotrexate (Rheumatrex) in patients, M. Gopal Nair and O. Castaneda observed a significant percentage (40%) of patients who were methotrexate non-responders. In addition, some patients also exhibited significant drug related toxicity including elevation of liver enzymes. In order to unravel the mechanism of methotrexate non-responsiveness M. G. Nair investigated the metabolism of methotrexate and its potential deactivation pathways. This investigation revealed that methotrexate and similar antifolate compounds underwent oxidative deactivation mediated by the enzyme aldehyde oxidase [Cellular Pharmacology. 3; 29-34 (1996)]. Methotrexate was efficiently converted to its corresponding 7-hydroxymethotrexate losing its potent antifolate properties. The amount of aldehyde oxidase vary among patients and those patients with higher levels of this enzyme deactivate the drug efficiently resulting in enhanced methotrexate non-responsiveness relative to those patients with average levels of this enzyme. This oxidative deactivation takes place in significance during the passage of methotrexate through the liver after absorption from the gut. Since sublingual administration of methotrexate does bypass this initial major deactivation route, this invention, that is the administration of sublingual dosage form of methotrexate, effectively reduces the deactivation process and thereby enhances the efficacy of methotrexate. Methotrexate non-responsiveness is not envisioned to be a problem during the practice of this Invention.

Inter patient variability of aldehyde oxidase can manifest as unexpected methotrexate toxicity. In humans having average normal levels of aldehyde oxidase, administration of methotrexate by the oral route results in average levels of oxidative deactivation. The optimal drug dosage is based on the normal average levels of deactivation of methotrexate. However if the amount of aldehyde oxidase is significantly lower in a percentage of patients receiving this drug, the plasma levels of the active drug are significantly enhanced resulting in antifolate toxicity. Therefore the variability of the enzyme aldehyde oxidase in patients can manifest either as non-responsiveness (high aldehyde oxidase) or as enhanced toxicity (low aldehyde oxidase).

The present invention of sublingual dosage forms of methotrexate, thus addresses two significant medical problems in treating not only the crippling, disfiguring and painful disease rheumatoid arthritis but also those inflammatory and auto-immune diseases for which methotrexate is prescribed by the conventional oral dosage form.

### Example-6

The aldehyde oxidase mediated oxidation of methotrexate vs. certain metabolically inert antifolates was investigated by M. Gopal Nair. [Med. Chem. Research. 9:176-185, 1999]. This investigation revealed that methotrexate quickly and very efficiently is oxidized to the corresponding and inactive 7-hydroxymethotrexate. Substantial oxidative inactivation of methotrexate takes place during the first pass of the drug via the liver subsequent to absorption from the gut. Since the sublingual administration of methotrexate bypasses this significant oxidative deactivation the Invention and Teachings reported herein offer significant and substantial therapeutic advantage in the treatment of a number of serious, acute and chronic illness that affects a large number of human beings.

### Advantages of Sublingual Dosage Units of Methotrexate:

1. Bioavailability of methotrexate by sublingual or trans-dermal delivery is independent of observed variations in bioavailability of drugs from the gut secondary to GI abnormalities.
2. Bioavailability of methotrexate by sublingual or trans-dermal delivery is independent of variations in bioavailability secondary to pH changes.
3. Bioavailability of methotrexate by sublingual or trans-dermal delivery is independent of variations in bioavailability due to deficiencies of carriers that mediate absorption from the gut.
4. Bioavailability of methotrexate by sublingual or trans-dermal delivery is independent of variations in bioavailability due to unknown and unpredictable variations in the GI tract biochemical pharmacology of individual patients.
5. Bioavailability of methotrexate by sublingual or trans-dermal delivery is independent of variations in bioavailability secondary to differences in the amount of intestinal micro-flora.
6. Bioavailability of methotrexate by sublingual or trans-dermal delivery is independent of variations in bioavailability due to differences in drug metabolism mediated by microorganism in the GI tract.
7. Bioavailability of methotrexate by sublingual or trans-dermal delivery is independent of variations in bioavailability due to changes in enterohepatic circulation.
8. Bioavailability of methotrexate by sublingual or trans-dermal delivery is independent of variations in bioavailability due to instability of the oral dosage unit formulation of the drug in the GI tract.
9. Sublingual or trans-dermal delivery of methotrexate is more convenient for patients with swallowing difficulties.
10. Bioavailability of methotrexate by sublingual or trans-dermal delivery is independent of variations in bioavailability in patients prone to misadventure by swallowing more or less the number of tablets due to forgetfulness.
11. Bioavailability of methotrexate by sublingual or trans-dermal delivery is independent of variations in bioavailability secondary to dietary habits/restrictions/or taking other prescription drugs.
12. Bioavailability of antifolates by sublingual or trans-dermal delivery is independent of variations in bioavailability due to alcoholism or other related abnormalities.

Methotrexate can be formulated for sublingual administration by using any of the published standard sublingual tablet manufacturing procedures. Such procedures have been described and any minor variations of such manufacturing procedures may not undermine or cited as causative to invalidate or interfere in any way with this Primary Invention; that is “Sublingual Methotrexate and Methotrexate Patches”

In general, diseases that may be treated with sublingual formulation of methotrexate may include but are not limited to autoimmune diseases, inflammatory diseases and neoplastic diseases. Further, the neoplastic diseases may be comprised of both malignant and benign growths characterized by abnormal cellular proliferation. Sublingual formulations of methotrexate can also be made using the poly-γ-glutamates of methotrexate, especially with shorter glutamate chain length ranging from two to three glutamate residues for therapeutic use.

Sublingual solid or liquid oral dosage forms of methotrexate or its poly-γ-glutamates may be manufactured by well-known and existing technologies used by commercial manufacturers. Methotrexate may be formulated for sublingual administration by using methotrexate as such as the di-acid or as its salts (sodium salt, potassium salt, salts of any mono-valent, divalent (example: calcium) or trivalent metal (example: Aluminum), salts with acids such as hydrochloride, hydro bromide, acetate, trifluoroacetate, tartrate or in general salts with any weak or strong acids. The formulation may be buffered to stabilize the pH with standard buffering agents or combined with citric acid or tartaric acid to provide protons during disintegration of the sublingual dosage unit for vasodilatation to enhance sublingual absorption. The formulation may be engineered to permit rapid disintegration of the sublingual dosage unit of the antifolate within a few minutes and release of more than 80% of the antifolate to the buccal cavity during disintegration. The antifolate compound may be combined with cellulose, sorbitol, croscamelose-sodium, mannitol, fructose, flavors, gelatin, magnesium stearate, syloid, dicalcium phosphate, xylitol, microcrystalline cellulose and citric acid. One or more disaccharide may be used as fillers. Gelatin may be used as a binder and disintegrants may include but are not limited to sodium starch glycolate, starches and CLPVP. The sublingual formulation may either be a solid dosage form or sublingual liquid dosage form consisting of water, alcohol and mannitol, sorbitol or natural honey in different proportions to maximize the absorption. The liquid sublingual dosage form may be used as drops under the tongue for convenient delivery and rapid absorption.

### Chemical Structure of Methotrexate

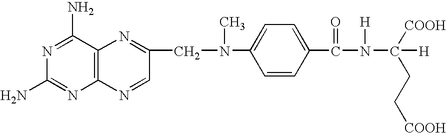

Pursuant to the preceding disclosures of invention, I claim:

1. A sublingual dosage form of methotrexate or its salt comprising of an amount that is therapeutically effective and nontoxic to ameliorate rheumatoid arthritis.

2. A sublingual dosage form of methotrexate or its salt comprising of an amount that is therapeutically effective and nontoxic to ameliorate atherosclerosis, cardiovascular disease or stroke.

3. A sublingual gel, liquid, solid or semi-solid dosage form of methotrexate or its salt comprising of an amount in the range of 0.1 milligram to 3,000 mg per dosage unit that is therapeutically effective and nontoxic to ameliorate a disease selected from a group consisting of: Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma, Occupational asthma; Exercise induced asthma; pediatric asthma; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodgkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Ulcerative Colitis, Immune thrombocytopenic purpuria, Evan's; Auto-immune hemolytic anemia; Vasculitis, Wegener's; Behcet's; Takayasu; Macroscopic poly. Nodosa; Classical poly. Nodosa; Churg-Strauss syndrome, Mysthenia Gravis, Leukemia, Lung cancer, Head and neck cancer, Brain tumor, Lymphoma, Choriocarcinoma, Ovarian cancer, Colon Cancer, Testicular cancer, Kidney cancer, Bladder Cancer, Breast cancer, Liver cancer, Stomach cancer, Coronary heart disease, Cardiovascular disease and stroke.

4. A patch impregnated or embedded with methotrexate in an amount that is therapeutically effective and nontoxic, in the range of 0.1 milligram to 3,000 milligram, of methotrexate that is worn over the skin of a human being or a warm blooded animal to ameliorate a disease that is selected from the group consisting of: Rheumatoid arthritis; Juvenile rheumatoid arthritis; Psoriasis; Psoriatic arthritis; Crohn's disease; Inflammatory bowel disease; Cystic fibrosis; Sarcoidosis; Ankylosing spondylitis [Pulmonary fibrosis, Amyloidosis], Multiple Sclerosis, Ulcerative colitis; Lupus erythematosis, Sjorgen syndrome; Reiter disease; Amyloidosis; Alzheimer's disease; Asthma, Occupational asthma; Exercise induced asthma; pediatric asthma; Uvitis; Scleritis; Orbital inflammation; Cryoglobulinemic vasculitis; Cryoglobulinemia; Immune thrombocytopenic purpuria (ITP); Myasthenia gravis; Autoimmune thyroiditis; Lupus nephritis; Steven Johnson syndrome; Sclerosing colangitis; Primary bilary cirrhosis; Cholestatic liver disease; Lambert Eaton myasthenic syndrome (LEMS); Vasculitides; Otitis; Rhinitis; Chronic obstructive pulmonary disease (COPD); Iritis; Iridocyclitis; Conjunctivitis; Neuromytotonia; Isaacs syndrome; Dyscrasiasis; Atopic dermatitis; Graft vs. Host disease (GVHD); Allograft rejection; Contact dermatitis; Churg-Strauss syndrome; Urticaria pigmentosa; Idiopathis hyper eosinophilic syndrome; Angio edema; Toxic epidermal necrolysis; Extrinsic allergic alveolitis (HP); Gluten sensitive enteropathy; Celiac disease; Sinusitis; Rhinosinusitis; Nasal polyposis; Laryngeal disorders; Peanut allergy; Eosinophillic esophagitis; Photo allergy; photo toxicity; Serum sickness reaction; Aspirin exacerbated respiratory disease; Hypersensitivity Pneumonitis; Chronic cough; Insect hypersensitivity; Anaphylaxis; Reactive mastocytosis; Atopic eczema; Autoantibody mediated blistering disease; Pemphigus; Bulles impetigo; Scalded skin syndrome; Autoimmune bullous disease; Allergic bronco pulmonary aspergillosis; Multiple myeloma; B-cell lymphoma; T-cell non-Hodgkin lymphoma; Primary cutaneous lymphoma; Monocytic leukemia; Systemic monocytosis; SSc; SLE; PM-DM; Sjogren's; Juvenile chronic arthritis; JIA; Other JIA; Polyarticular JIA; Ulcerative Colitis, Immune thrombocytopenic purpuria, Evan's; Auto-immune hemolytic anemia; Vasculitis, Wegener's; Behcet's; Takayasu; Macroscopic poly. Nodosa; Classical poly. Nodosa; Churg-Strauss syndrome, Mysthenia Gravis, Leukemia, Lung cancer, Head and neck cancer, Brain tumor, Lymphoma, Choriocarcinoma, Ovarian cancer, Colon Cancer, Testicular cancer, Kidney cancer, Bladder Cancer, Breast cancer, Liver cancer, Stomach cancer, Atherosclerosis, Coronary heart disease, Cardiovascular disease and stroke.
